(19) United States
(12) Patent Application Publication
(10) Pub. No.: US 2024/0033304 A1
(43) Pub. Date: Feb. 1, 2024

(54) COMPOSITION FOR PREVENTING OR TREATING BRAIN TUMOR IN HIGH-GLUCOSE ENVIRONMENT

(71) Applicant: KOREA ADVANCED INSTITUTE OF SCIENCE AND TECHNOLOGY, Daejeon (KR)

(72) Inventors: Heung Kyu Lee, Daejeon (KR); Jaeho Kim, Daejeon (KR)

(21) Appl. No.: 18/258,643

(22) PCT Filed: Dec. 28, 2021
(86) PCT No.: PCT/KR2021/020049

§ 371 Date: Jun. 21, 2023

(30) Foreign Application Priority Data
Dec. 29, 2020 (KR) 10-2020-0186828

(51) Int. Cl. A61K 35/74; A61K 39/395; A61P 35/00

(52) U.S. Cl. CPC A61K 35/74; A61K 39/3955; A61P 35/00; A61K 2035/11

(57) ABSTRACT

The composition of the present invention induces changes in the composition of gut microbiota, particularly changes in the composition of Desulfovibrionasaceae strains, in a high glucose diet environment, so it has no toxicity to the body and has excellent brain tumor inhibitory activity, and thus it can effectively prevent, improve or treat brain tumors. Furthermore, such changes in the composition of gut microbiota in a high glucose diet environment induces a synergistic effect with anticancer drugs, particularly immune checkpoint inhibitors, and thus it can ultimately contribute to improving the survival rate of brain tumor patients.

### CROSS-REFERENCE TO RELATED APPLICATIONS

This application is a U.S. National Stage entry of International Patent Application no. PCT/KR2021/020049, filed Dec. 28, 2021, which claims the benefit of priority of Korean Patent Application no. 10-2020-0186828, filed Dec. 29, 2020.

### TECHNICAL FIELD

The present invention relates to a composition for preventing or treating brain tumor using gut microbiota in a high glucose-intake environment.

### BACKGROUND OF THE INVENTION

Recently, environmental hygiene has improved and the risk of infectious diseases in humans has rapidly decreased due to the development of vaccines and antibiotics, while the extension of life due to improved quality of life has brought about other problems such as cardiovascular diseases, metabolic diseases, and malignant tumors. In infectious diseases, the risk of these diseases increases not due to a single cause, such as a pathogen, but a combination of various factors, and one of those factors, Westernized eating habits, has been found to increase the incidence of the disease. Westernized eating habits refer mainly to the intake of foods, such as fast food or carbonated beverages, which contain excessive amounts of saturated fat, sugar, and salt. In particular, frequent intake of high-sugar drinks, especially carbonated drinks, is associated with metabolic disorders such as obesity. Although many studies have already been conducted regarding these problems in the Western world, studies on how these eating habits affect the immune system or the development or progression of malignant tumors are still insufficient.

Recently, it has been confirmed through animal models that the intake of high-sugar beverages increases the severity of autoimmune diseases. In addition, studies in the colon cancer model have shown that the intake of high-sugar drinks promote tumor growth, suggesting the possibility that high-sugar drinks can not only cause metabolic diseases such as obesity, but also affect anticancer immune responses. However, there is no research on the effect of high glucose intake on antitumor immune responses, and the effect of high glucose intake on antitumor immune responses particularly in relation to brain tumors is not yet known, so such research is necessary.

On the other hand, microbiota, which refers to all microorganisms that live symbiotically in the human body, refers to a microbial community comprising bacteria, archaea, and eukarya that exist in a specific environment, and gut microbiota is known to play an important role in human physiology and has a great influence on human health and disease through interaction with human cells. While it is predicted that a high glucose-intake can induce changes in intestinal symbiotic microorganisms and affect the immune response, research on the relationship between high glucose intake, intestinal microorganisms, and cancer occurrence is insufficient.

Therefore, the present inventors used a mouse model using a high-sugar beverage to confirm changes in the intestinal microorganisms caused by high glucose intake, and confirmed the positive effect of changes in specific microorganisms among the intestinal microorganisms on the anti-brain tumor immune response.

### DISCLOSURE OF THE INVENTION

### Technical Problem

One objective of the present invention is to provide a pharmaceutical composition for preventing, alleviating or treating brain tumor in subjects with high glucose-intake.

Another objective of the present invention is to provide a method for preventing or treating brain tumor, comprising administering, to a subject in need thereof, a pharmaceutically effective amount of a composition comprising at least one selected from the group consisting of the Desulfovibrionaceae family strain, cultures thereof, culture mediums, and extracts obtained therefrom as an active ingredient.

Still another objective of the present invention is to provide a food composition for preventing, alleviating or treating brain tumor in subjects with high glucose-intake.

However, the technical problem to be achieved by the present invention is not limited to the above-mentioned problems, and other problems not mentioned will be clearly understood by those skilled in the art from the following detailed description.

### Technical Solution

Hereinafter, various embodiments described herein are described with reference to the drawings. In the following description, various specific details, such as specific forms, compositions and processes, etc., are set forth in order to provide a thorough understanding of the present invention. However, certain embodiments may be executed without one or more of these specific details, or with other known methods and forms. In other instances, well known processes and manufacturing techniques are not described in specific detail to avoid unnecessarily obscuring the present invention. References throughout this specification to “one embodiment” or “an embodiment” means that a particular feature, form, composition or characteristic described in relation to the embodiments is included in one or more embodiments of the present invention. Therefore, the appearances of “in one embodiment” or “an embodiment” in various places throughout this specification do not necessarily refer to the same embodiment of the present invention. In addition, particular features, forms, compositions, or characteristics may be combined in one or more embodiments in any suitable way.

Unless there is a specific definition within the specification, all scientific and technical terms used herein have the same meaning as commonly understood by a person skilled in the art to which the present invention belongs.

One embodiment of the present invention relates to a pharmaceutical composition for preventing, alleviating or treating brain tumor.

The pharmaceutical composition of the present invention may comprise at least one selected from the group consisting of the Desulfovibrionaceae family strain, cultures thereof, culture broths, and extracts obtained therefrom, as an active ingredient.

In the present invention, the “Desulfovibrionaceae family strain” as used herein is included in the class of Deltaproteobacteria consisting of Desulfovibrionaceae and desulfobacteriaceae, and the Desulfovibrionasae family currently consists of three genera with valid names: Desulfovibrio, Bilophila and Lawsonia. The Desulfovibrio refers to the name of a representative genus comprising a bacterial species that uses sulfate as an electron acceptor. Bacterial species of the Disulfovibrio exhibit the characteristics of obligate anaerobe bacteria, a curved rod shape and motility, and are known to live in various environments such as rivers, lakes, oceans, soils, and animal intestines. In particular, these bacteria are often found in contaminated environments for they are known to oxidize various organic substances by reducing sulfide substances such as sulfate to hydrogen sulfide under anaerobic conditions, and to affect heavy metal toxicity changes. Desulfovibrio, like other sulfate-reducing bacteria, has long been recognized as an obligatory anaerobe, but since it can withstand an oxygen-rich environment, it has recently been classified as an aerotolerant. In the case of strains of the genus Bilophila, they are classified as harmful bacteria in the intestines because it is involved in the production of hydrogen sulfide in the intestines, and hydrogen sulfide is known to cause colorectal cancer, while intracellularis of the strains of the genus Lawsonia is also known as a bacterium that causes pig disease. In the present invention, strains of the Desulfovibrionaceae family may comprise strains belonging to genus such as Desulfovibrio, Bilophila and Lawsonia, but are not limited thereto.

In the present invention, stains belonging to the Desulfovibrio genus may be at least one selected from the group consisting of Desulfovibrio vulgaris (D. vulgaris), Desulfovibrio acrylicus (D. acrylicus), Desulfovibrio aerotolerans (D. aerotolerans), Desulfovibrio aespoensis (D. aespoeensis), Desulfovibrio africanus (D. africanus), Desulfovibrio alaskensis (D. alaskensis), Desulfovibrio alcoholivorans (D. alcoholivorans), Desulfovibrio alkalitolerans (D. alkalitolerans), Desulfovibrio aminophilus (D. aminophilus), Desulfovibrio arcticus (D. arcticus), Desulfovibrio barsi (D. baarsii), Desulfovibrio baculatus (D. baculatus), Desulfovibrio bastini (D. bastinii), Desulfovibrio biadensis (D. biadhensis), Desulfovibrio bizertensis (D. bizertensis), Desulfovibrio burkinensis (D. burkinensis), Desulfovibrio butyratiphilus (D. butyratiphilus), Desulfovibrio capillatus (D. capillatus), Desulfovibrio carbinolicus (D. carbinolicus), Desulfovibrio carbinoliphilus (D. carbinoliphilus), Desulfovibrio cuneatus (D. cuneatus), Desulfovibrio dechloroacetivorans (D. dechloracetivorans), Desulfovibrio desulfuricans (D. desulfuricans), Desulfovibrio perirreducens (D. ferrireducens), Desulfovibrio frigidus (D. frigidus), Desulfovibrio fructosivorans (D. fructosivorans), Desulfovibrio furfuralis (D. furfuralis), Desulfovibrio gabonensis (D. gabonensis), Desulfovibrio giganteus (D. giganteus), Desulfovibrio gigas (D. gigas), Desulfovibrio gracilis (D. gracilis), Desulfovibrio halophilus (D. halophilus), Desulfovibrio hydrothermalis (D. hydrothermalis), Desulfovibrio idahonensis (D. idahonensis), Desulfovibrio indonesiensis (D. indonesiensis), Desulfovibrio inopinatus (D. inopinatus), Desulfovibrio internalis (D. intestinalis), Desulfovibrio legallii (D. legallii), Desulfovibrio alitoralis (D. alitoralis), Desulfovibrio longreachensis (D. longreachensis), Desulfovibrio longus (D. Iongus), Desulfovibrio magneticus (D. magneticus), Desulfovibrio marinus (D. marinus), Desulfovibrio marinisediminis (D. marinisediminis), Desulfovibrio marrakechensis (D. marrakechensis), Desulfovibrio mexicanus (D. mexicanus), Desulfovibrio multispirans (D. multispirans), Desulfovibrio oceani (D. oceani), Desulfovibrio oxamicus (D. oxamicus), Desulfovibrio oxyclinae (D. oxyclinae), Desulfovibrio paquesii (D. paquesii), Desulfovibrio piezophilus (D. piezophilus), Desulfovibrio pigra (D. pigra), Desulfovibrio portus (D. portus), Desulfovibrio profundus (D. profundus), Desulfovibrio psychrotolerans (D. psychrotolerans), Desulfovibrio putealis (D. putealis), Desulfovibrio salixigens (D. salixigens), Desulfovibrio sapovorans (D. sapovorans), Desulfovibrio senezii (D. senezii), Desulfovibrio simplex (D. simplex), Desulfovibrio sulfodismutans (D. sulfodismutans), Desulfovibrio termitidis (D. termitidis), Desulfovibrio thermophilus (D. thermophilus), Desulfovibrio tunisiensis (D. tunisiensis), Desulfovibrio vietnamensis (D. vietnamensis) and Desulfovibrio zosterae (D. zosterae), preferably Desulfovibrio vulgaris (D. vulgaris), but is not limited thereto.

The term “culture” as used herein refers to a product obtained by culturing a microorganism in a known liquid or solid medium, and refers to a medium containing the microorganism.

The culture of the present invention is obtained after culturing the strain of the present invention in a medium, and the medium may be selected from a known liquid or solid medium used to culture Desulfovibrionasae family strains, such as GYSM medium, humic acid agar medium, Bennett's agar medium, or malt extract agar medium, but is not limited thereto.

The term “culture medium” as used herein refers to a product obtained by culturing microorganisms in a known liquid or solid medium, and refers to a concept that does not contain microorganisms. The culture medium of the present invention refers to a liquid product from which the strain itself is removed through methods such as filtration or centrifugation after culturing a given strain in a liquid medium, and more concretely, the culture medium of the present invention refers to the culture of the strain belonging to the Desulfovibrionaceae family from which the strain is removed.

The filtration method of the present invention is not particularly limited, and the number of filtrations performed may be, for example 1 to 10 times, 1 to 5 times or 1 to 3 times. In addition, when filter paper or a filter is used for the filtration, the pore size of the filter paper may be 5 to 10 μm or a filer with a pore size of 0.1 to 1.0 μm may be used, but is not limited thereto

The term “extract” as used herein may be a culture or extract of culture medium of a strain belonging to the Desulfovibrionaceae family, and can be prepared using conventional extraction methods known in the art, such as solvent extraction, extraction by supercritical fluid extraction using carbon dioxide, extraction by ultrasonication, separation using an ultrafiltration membrane with a constant molecular weight cut-off value, various chromatography (made for separation according to size, charge, hydrophobicity or affinity), or a combination thereof.

The extraction solvent used in the solvent extraction method of the present invention may be at least one selected from the group consisting of water, a lower alcohol having 1 to 4 carbon atoms (eg. methanol, ethanol, propanol and butanol), or a hydrous lower alcohol that is a mixture thereof, propylene glycol, 1,3-butylene glycol, glycerin, acetone, diethyl ether, ethyl acetate, butyl acetate, dichloromethane, chloroform, hexane and mixtures thereof, and may be selected from water, alcohol, hydrous alcohol, diethyl ether, ethyl acetate, butyl acetate, chloroform or hexane, but is not limited thereto.

The strain, its culture, its culture medium or an extract obtained therefrom of the present invention can be used for preventing or treating brain tumor by inducing changes in the composition of the gut microbiota in a subject with high glucose-intake.

In the present invention, the term “high glucose-intake” refers to a diet that consumes a large amount of sugars exceeding 50 g, calculated by converting 10% (200 kcal) of the 2000 kcal daily caloric intake recommended by the World Health Organization (WHO) for adults to daily sugar intake, and thus refers to the intake of sugars in excess of 10% of the total caloric intake. According to the Ministry of Food and Drug Safety, the daily average of sugar intake of Koreans is 61.4 g, which exceeds the recommended amount, and numerous studies have shown that excessive consumption of sugar in excess of the recommended amount increases the risk of various diseases comprising obesity, type 2 diabetes, cardiovascular disease and cancer. According to the Korean Society of Nutrition, sugar intake standards should be evaluated based on the contribution ratios of individuals to their daily energy intake rather than an absolute intake standard. Thus in the present invention, the standard of high glucose-intake refers to intake exceeding 0.02 g/kcal to 0.025 g/kcal as a ratio of weight (g) of total intake of sugars to total calories (kcal) per day (24 hours). Concretely, it may refer to intake exceeding 0,023 g/kcal.

The term “gut microbiota” as used herein refers to a microbial community comprising bacteria, archaea and eukarya present in the intestine of subjects. Gut microbiota plays an important role in human physiology and has a great influence on health and disease of human through interaction with human cells. In particular, there are 100 trillion microorganisms in symbiosis with the human body, which is 10 times more than human cells, and the number of microorganism genes is more than 100 times the number of human genes. Bacteria that live in symbiosis with our body secrete nanometer-sized vesicles to exchange information such as genes and proteins with other cells. The mucous membrane forms a physical barrier that does not allow particles larger than 200 nanometers (nm) to pass through, so bacteria living in symbiosis on the mucous membrane cannot pass the membrane, but bacteria-derived vesicles are usually less than 100 nanometers in size, making it relatively easy for them to pass through mucous membranes and absorb into our body. Recently, various studies have been conducted on the effect of changes in intestinal microorganisms on the immune response.

The composition of the present invention may further comprise an immune checkpoint inhibitor.

The term “immune checkpoint inhibitor” as used herein refers to a drug that activates T cells to attack cancer cells by blocking the activity of immune checkpoint proteins involved in T cell suppression. In other words, cancer cells have an evasion mechanism that avoids attack by expressing PD-L1 and causing immune cells to recognize them as nonself, but the drug suppresses immune response evasion signals, allowing immune cells to attack cancer cells so that cancer cells can be eliminated. New drugs developed to neutralize the immune system evasion response of cancer cells typically target sites such as CTLA-4, PD-1, PD-L1, PD-L2, LAG-3, TIM-3, BTLA or KIR, and use substances that specifically bind to these proteins. Here, the term substance above corresponds to a broad concept including antibodies, but is not limited thereto.

The immune checkpoint inhibitor of the present invention may be at least one selected from the group consisting of a CTLA-4 receptor inhibitor, a PD-1 receptor inhibitor, a PD-L1 ligand inhibitor, a PD-L2 ligand inhibitor, a LAG-3 receptor inhibitor, a TIM-3 receptor inhibitor, a BTLA receptor inhibitor and a KIR receptor, and more concretely may be a PD-1 receptor inhibitor, but is not limited thereto.

The “PD-1 receptor inhibitor” of the present invention inhibits the immune evasion function of cancer cells by binding to the PD-1 receptor of T cells. When a protein on the surface of cancer cells or hematopoietic cells called PD-L1, also called CD274 or B7-H1, is expressed on the surface of specific cells, it binds to PD-1, a protein on the surface of T cells, and inhibits T cell function, making it unable to attack cancer cells. In the case of a PD-1 inhibitor, when a PD-1 monoclonal antibody binds to PD-1, T cells that were suppressed are activated to attack and kill cancer cells. Unlike conventional immunotherapeutic agents (cytokine therapeutics, anticancer vaccines), it binds to the binding site of cancer cells and T cells to block immune evasion signals, thereby preventing the formation of immunological synapses, and has a mechanism that allows T cells that are not interfered by immune evasion to destroy cancer cells. In other words, although cancer cells have an immune evasion substance called PD-L1 and proliferates by inactivating immune cells, when the PD-1 antibody first binds to PD-L1 and the PD-1 binding site, immune evasion signals are blocked and T cells that do not receive these signals kill cancer cells. The PD-1 receptor inhibitor targets the PD-1/PD-L1 pathway a protein expressed in immune cells and cancer cells in the body, blocks their mutual relationship, and helps immune cells attack cancer cells.

When the composition of the present invention is used in combination with the immune checkpoint inhibitor in a high glucose-intake environment, a synergistic effect can be induced in the treatment of brain tumor.

The term “brain tumor” as used herein refers to all tumors generated within the cranial cavity, and depending on the location of occurrence, even benign tumors may have poor prognosis like malignant tumors, and have a higher risk of recurrence than cancers occurring in other organs. These brain tumors can be divided into two types: primary brain tumors and metastatic brain tumors, wherein primary brain tumors originate from the brain itself and metastatic brain tumors means they have spread to the brain from other organs. Glioblastoma multiforme, a type of brain cancer, is a malignant tumor with a high degree of malignancy. According to the WHO classification, it corresponds to the most dangerous level, stage 4, and even with a combination of surgery, chemotherapy and radiotherapy, the 5-year or longer survival rate barely exceeds 1%. On average, the survival rate of cancer patients has improved by about 30%, but in the case of glioblastoma the survival rate is maintained at a remarkably low level of 1% due to the characteristics of the organ. In the case of the brain which is known as an immune-tolerant organ, while the blood-brain barrier (BBB) allows for the passage of immune cells under certain circumstances, there are still significant limitations in the use of immune checkpoint inhibitors, which are the third-generation anticancer drugs. Therefore, since treatment of brain tumor is very difficult, it is very important to diagnosis is made at an early stage compared to other carcinomas in order to deal with it efficiently. However, despite its importance, it is impossible to diagnose brain tumors early through the general process of visiting the hospital after the patient himself recognizes its symptoms, since only common symptoms that can be experienced in daily life, such as headache or nausea, appear, especially in the case of glioblastoma. Magnetic Resonance Imaging (MRI) is a representative diagnostic method for brain tumors such as glioblastoma. However, since this MRI imaging method is very expensive there is a limitation that the entry barriers are very high to be applied to cases with a low risk of developing a disease. Furthermore, in the case of MRI imaging, a metal contrast agent is administered into the body, so it is not applicable when there is an allergic reaction to such a contrasting agent, but such metal may accumulate in the body when taking multiple scans and cause kidney diseases.

The term “brain tumor” as used herein refers to both primary tumors arising from brain tissue or membranes surrounding the brain, and metastatic tumors in which tumors metastasize from the skull or surrounding structures, or an area distant from the head, to brain tissues or meningeal membranes. For the purposes of the present invention, the brain tumor may be glioblastoma, but is not limited thereto.

The “glioblastoma” of the present invention is a tumor with the highest degree of malignancy among tumors arising from glial cells in the brain, in which nuclear atypia, mitotic phase, vascular endothelial cell proliferation and necrosis are histologically observed. Malignant glioblastoma accounts for 12 to 15% of all brain tumors and 50 to 60% of brain gliomas, and is the most common single tumor that occurs in the brain.

The term “prevention” as used herein refers to any act utilizing therapy to protect against the onset of brain tumor diseases using the composition of the present invention, and more concretely, to prevent the onset, recurrence or spread of brain tumor symptoms in subjects with high glucose.

The term “alleviation” as used herein refers to blocking brain tumor symptoms using the composition of the present invention, and more concretely comprises, without limitation, any activity that suppresses or delays brain tumor symptoms in subjects with high glucose.

The term “treatment” as used herein refers to a series of activities performed to alleviate or improve brain tumors using the composition of the present invention, and more concretely may comprise, without limitation, all activities that improve or benefit brain tumors in subjects with high glucose.

In the present invention, the pharmaceutical composition may be in the form of capsules, tablets, granules, injections, ointments, powders or beverages, and the pharmaceutical composition may be for humans.

The pharmaceutical composition is not limited to such, but can be formulated according to conventional methods into oral formulations such as powders, granules, capsules, tablets and aqueous suspensions, and into external preparations, suppositories and sterile injection solutions. The pharmaceutical composition of the present invention may comprise a pharmaceutically acceptable carrier. The pharmaceutically acceptable carriers may comprise binders, lubricants, disintegrants, excipients, solubilizers, dispersants, stabilizers, suspending agents, pigments, flavors, and the like for oral administrations, buffers, preservatives and painless agents for injections, and bases, excipients, lubricants, preservatives and the like for topical administrations. The formulation of the pharmaceutical composition of the present invention may be diversely prepared by mixing with a pharmaceutically acceptable carrier as noted above. For example, the pharmaceutical composition may be prepared in the form of tablets, troches, capsules, elixirs, suspensions, syrups, wafers and the like for oral administration, and in unit dosage ampoules or multiple dosage forms for injections. In addition, it may be formulated into solutions, suspensions, tablets, capsules, sustained-release preparations, and the like.

On the other hand, examples of carriers, excipients and diluents suitable for formulation can be lactose, dextrose, sucrose, sorbitol, mannitol, xylitol, erythritol, malditol, starch, acacia gum, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate or mineral oil, and the like. In addition, it may also comprise fillers, anti-coagulants, lubricants, wetting agents, flavoring agents, emulsifiers, preservatives, and the like.

The route of administration of the pharmaceutical composition of the present invention may comprise, but is not limited to, oral, intravenous, intramuscular, intraarterial, intramedullary, intrathecal, intracardiac, transdermal, subcutaneous, intraperitoneal, intranasal, intestinal, topical, sublingual or rectal. Oral or parenteral administration is preferred.

The term “parenteral” as used herein comprises subcutaneous, intradermal, intravenous, intramuscular, intraarticular, intracapsular, intrasternal, intrathecal, intralesional and intracranial injection or infusion techniques. The pharmaceutical composition of the present invention may also be administered in the form of a suppository for rectal administration.

The pharmaceutical composition of the present invention may change depending on various factors including the activity of the specific compound used, age, body weight, general health, sex, diet, administration time, route of administration, excretion rate, drug combination and severity of the specific disease to be prevented or treated. The dosage of the pharmaceutical composition of the present invention varies, but can be appropriately determined by those skilled in the art depending on the patient's condition, weight, disease severity, drug type, administration route and period, and can be administered at 0.0001 to 50 mg/kg or 0.001 to 50 mg/kg per day. Administration can be administered once a day, or may be administered in several divided dosages. The dosage is not intended to limit the scope of the present invention in any way. The pharmaceutical composition of the present invention may be formulated into a pill, dragee, capsule, liquid, gel, syrup, slurry, or suspension.

Another embodiment of the present invention relates to a method for preventing or treating brain tumor comprising the step of administering a pharmaceutically effective amount of a composition comprising at least one selected from the group consisting of the Desulfovibrionaceae family strain, cultures thereof, culture mediums, and extracts obtained therefrom as an active ingredient, to a subject in need of administration.

In the method for preventing or treating brain tumor of the present invention, descriptions of the Desulfovibrionaceae family strain, cultures thereof, culture mediums, extracts obtained therefrom, and brain tumors are the same as those described in the pharmaceutical composition for preventing, alleviating or treating brain tumor, and is thus descriptions thereof are omitted to avoid excessive redundancy.

The term “administration” as used herein refers to providing a predetermined composition of the present invention to a subject by any suitable method.

The term “subject” in need of the administration as used herein may comprise both mammals and non-mammals. Here, examples of mammals may comprise humans, non-human primates such as chimpanzees, other apes or money species, livestock animals such as cattle, horses, sheep, goats, pigs, domesticated animals such as rabbits, dogs or cats, as well as laboratory animals such as rodents, such as rats, mice or guinea pigs, but are not limited thereto. In addition, examples of non-mammals in the present invention may comprise birds or fish, but are not limited thereto.

In the present invention, the formulation of the composition administered as described above are not particularly limited, and may be administered as a solid formulation, liquid formulation, or aerosol formulation for inhalation, as a solid formulation intended to be converted into a liquid form preparation for oral or parenteral administration immediate prior to usage, as oral formulations such as powders, granules, capsules, tablets and aqueous suspensions, and as external preparations, suppositories and sterile injection solutions, but is not limited thereto.

In addition, in the present invention a pharmaceutically acceptable carrier may be additionally administered with the formulation of the present invention during the administration. Here, the pharmaceutically acceptable carrier may be binders, lubricants, disintegrants, excipients, solubilizers, dispersants, stabilizers, suspending agents, pigments, flavors, and the like for oral administration, a mix of buffers, preservatives, analgesic agents, solubilizers, isotonic agents, stabilizers, and the like for injections, and bases, excipients, lubricants, preservatives, and the like for topical administration. The formulation of the composition of the present invention may be diversely prepared by mixing with a pharmaceutically acceptable carrier as noted above. For example, the pharmaceutical composition may be prepared in the form of tablets, troches, capsules, elixirs, suspensions, syrups, wafers and the like for oral administration, and in unit dosage ampoules or multiple dosage forms for injections. In addition, it may be formulated into solutions, suspensions, tablets, capsules, sustained-release preparations, and the like.

On the other hand, examples of carriers, excipients and diluents suitable for formulation can be lactose, dextrose, sucrose, sorbitol, mannitol, xylitol, erythritol, malditol, starch, acacia gum, alginate, gelatin, calcium phosphate, calcium silicate, Cellulose, methyl cellulose, microcrystalline cellulose, polyvinylpyrrolidone, water, methylhydroxybenzoate, propylhydroxybenzoate, talc, magnesium stearate or mineral oil, and the like. In addition, it may also comprise fillers, anti-coagulants, lubricants, wetting agents, flavoring agents, emulsifiers, preservatives, and the like.

The route of administration of the pharmaceutical composition of the present invention may comprise, but is not limited to, oral, intravenous, intramuscular, intraarterial, intramedullary, intrathecal, intracardiac, transdermal, subcutaneous, intraperitoneal, intranasal, intestinal, topical, subglossal or rectal. Oral or parenteral administration is preferred.

The term “parenteral” as used herein comprises subcutaneous, intradermal, intravenous, intramuscular, intraarticular, intracapsular, intrasternal, intrathecal, intralesional and intracranial injection or infusion techniques. The pharmaceutical composition of the present invention may also be administered in the form of a suppository for rectal administration.

The term “pharmaceutically effective amount” as used herein refers to a sufficient amount of an agent to provide a desired biological result. The result may be reduction and/or alleviation of the signs, symptoms, or causes of a disease, or any other desirably alteration of a biological system. For example, an “effective amount” for therapeutic use is the amount of a protein or gene disclosed in the present invention required to provide a clinically significant reduction in disease. An “effective” amount suitable in any individual case can be determined by one skilled in the art using routine experimentation. Thus, the expression “effective amount” generally refers to an amount of an active substance that has a therapeutic effect. In the case of the present invention, the active substance is a formulation of the composition comprising at least one selected from the group consisting of strains of the Desulfovibrionaceae family, cultures thereof, culture mediums and extracts obtained therefrom, and a preventative or therapeutic agent for brain tumor.

The formulation of the present invention may change depending on various factors including the activity of the Desulfovibrionaceae family strain used, individual's age, body weight, general health, sex, diet, administration time, route of administration, excretion rate, drug combination and severity of the specific disease to be prevented or treated. The dosage of the pharmaceutical composition of the present invention varies, but can be appropriately determined by those skilled in the art depending on the patient's condition, weight, disease severity, drug type, administration route and period, and can be administered at 0.0001 to 100 mg/kg or 0.001 to 100 mg/kg per day. Administration can be administered once a day, or may be administered in several divided dosages. The dosage is not intended to limit the scope of the present invention in any way. The pharmaceutical composition of the present invention may be formulated into a pill, dragee, capsule, liquid, gel, syrup, slurry, or suspension.

The formulation of the present invention can be used alone, or in combination with surgery, radiation therapy, hormone therapy, chemotherapy, and methods using biological response modifiers.

In addition, the formulation of the present invention can be used in combination with other anticancer agents, wherein the anticancer agent is at least one selected from the group consisting of nitrogen mustard, imatinib, oxaliplatin, rituximab, erlotinib, neratinib, lapatinib, gefitinib, vandetanib, nirotinib, semasanib, bosutinib, axitinib, cediranib, lestaurtinib, Trastuzumab, gefitinib, bortezomib, sunitinib, carboplatin, sorafenib, bevacizumab, cisplatin, cetuximab, viscum album, asparaginase, tretinoin, hydroxycarbamide, dasatinib, estramustine, gemtuzumab ozogamicin, ibritumomab tucetan, heptaplatin, methyl aminolevulinic acid, amsacrine, alemtuzumab, procarbazine, alprostadil, holmium nitrate chitosan, gemcitabine, Doxifluridine, pemetrexed, tegafur, capecitabine, gimeracil, oteracil, azacytidine, methotrexate, uracil, cytarabine, fluorouracil, fludarabine, enocitabine, flutamide, capecitabine, decitabine, mercaptopurine, thioguanine, cladribine, carmophor, raltitrexed, docetaxel, paclitaxel, irinotecan, belotecan, topotecan, vinorelbine, etoposide, vincristine, vinblastine, teniposide, doxorubicin, idarubicin, epirubicin, mitoxantrone, mitomycin, bleromycin, daunorubicin, dactinomycin, pirarubicin, aclarubicin, pepromycin, Tensirolimus, temozolomide, busulfan, ifosfamide, cyclophosphamide, melphalan, altretamine, dacarbazine, thiotepa, nimustine, chlorambucil, mitolactol, leucovorin, tretonin, exemestane, aminoglutethimide, anagrelide, olaparib, navelbine, fadrozole, tamoxifen, toremifene, testolactone, anastrozole, letrozole, vorozole, bicalutamide, lomustine, vorinostat, entinostat, phenformin, metformin, talazoparib, and carmustine, but is not limited thereto.

Still another embodiment of the present invention relates to a food composition for preventing or alleviating brain tumor.

The food composition of the present invention may comprise, as an active ingredient, at least one selected from the group consisting of Desulfovibrionaceae family strains, cultures thereof, culture mediums, and extracts obtained therefrom.

The strains, cultures thereof, culture mediums, and extracts obtained therefrom may be used to prevent or alleviate brain tumor by inducing compositional changes in the gut microbiota in subjects with high glucose intake.

In the food composition of the present invention, descriptions of the Desulfovibrionaceae family strain, cultures, culture mediums, extracts, individuals with high glucose intake and brain tumors are the same as those described in the pharmaceutical composition, and is thus descriptions thereof are omitted to avoid excessive redundancy.

In the present invention, the food composition is used variously for the prevention or improvement of the target indication in the present invention, and may be manufactured in the form of various foods, such as beverages, chewing gum, tea, vitamin complexes, powders, granules, tablets, capsules, confectionary, rice cakes, and bread. The food composition of the present invention can be safely used even for long periods of time for preventive purposes because it is improved and constructed of existing food intakes with little toxicity and side effects. When the composition of the present invention is included in the food composition, it may be added at a ratio of 0.1 to 100% of the total weight. Here, when the food composition is manufactured in the form of a beverage, there is no particular limitation except that the food composition is contained in the indicated ratio, and various flavoring agents or natural carbohydrates may be included as additional ingredients as in conventional beverages. In other words, monosaccharides such as glucose, disaccharides such as fructose, polysaccharides such as sucrose, and common sugars such as dextrin and cyclodextrin, and sugar alcohols such as xylitol, sorbitol, and erythritol can be comprised as natural carbohydrates. Examples of the flavoring agent may comprise natural flavoring agents (thaumatin, stevia extract (eg. rebaudioside A, glycyrrhizin, etc.) and synthetic flavoring agents (saccharin, aspartame, etc.). In addition, the food composition of the present invention may contain various nutrients, vitamins, minerals (electrolytes), flavors such as synthetic flavors and natural flavors, colorants, pectic acid and its salts, alginic acid and its salts, organic acids, protective colloidal thickeners, pH regulators, stabilizers, preservatives, glycerin, alcohol, carbonation agent used in carbonated beverages, and the like. These components may be used independently or in combination. The ratio of these additives is generally selected from the range of 0.1 to 100 parts by weight per 100 parts by weight of the composition of the present invention, but is not limited thereto.

### Effects of the Invention

The present invention can effectively prevent, alleviate, or treat brain tumor diseases by inducing changes in the composition of the gut microbiota of subjects with high glucose-intake.

### BRIEF DESCRIPTION OF THE DRAWINGS

FIGS. 1A and 1B represent changes in survival rate and body weight in each group of mice fed normal drinking water as a control group, mice fed a high-glucose drink after cancer transplantation, and mice fed a high-glucose drink for 5 weeks before cancer transplantation using brain tumor mouse model according to an embodiment of the present invention.

FIGS. 2A and 2B represent results comparing the change in survival rate after ingesting a high glucose drink for 2 or 4 weeks before cancer transplantation according to an embodiment of the present invention with that of a control group.

FIG. 2C represents the change in survival rate after ingesting a high glucose drink for 5 weeks to germ-free mice without intestinal symbiotic microorganisms according to an embodiment of the present invention.

FIG. 3 represents the analysis of the composition of symbiotic intestinal microbial community by performing 16S ribosomal DNA sequencing on DNA extracted from the feces of cancer-transplanted and non-transplanted groups in a brain tumor mouse model ingesting a high glucose beverage according to an embodiment of the present invention.

FIG. 4 represents the amount of change in intestinal symbiotic microorganisms at the family stage in terms of linear discriminant analysis size (LDA size) in each group transplanted with and not transplanted with cancer according to an embodiment of the present invention.

FIGS. 5A and 5B represent the inhibitory effect of brain tumor growth due to intake of Desulfovibrionaceae strains when high glucose drinks were supplied after dividing into groups that ingested bacteria from 2 weeks before transplanting cancer until 2 weeks after transplantation for a total of 4 weeks, and that ingested bacteria and supplied high glucose beverages at the same time, according to an embodiment of the present invention.

FIG. 6 represents the Uniform Manifold Approximation and Projection (UMAP) analysis of CD8+ T cells among all immune cells after single-cell RNA sequencing of immune cells extracted from brain tumors of the group that ingested high glucose beverages (HG) and the group that ingested normal drinking water (NW) according to an embodiment of the present invention.

FIGS. 7A and 7B represent changes in survival rates after intraperitoneal injection of anti PD-1 to the group that ingested high glucose beverages (HG) and the group that ingested normal drinking water (NW) according to an embodiment of the present invention.

### BEST MODE FOR IMPLEMENTATION OF THE INVENTION

One embodiment of the present invention relates to a pharmaceutical composition for preventing, alleviating or treating brain tumor in subjects on a high glucose diet.

Another embodiment of the present invention relates to a method for preventing or treating brain tumor comprising the step of administering a pharmaceutically effective amount of a composition comprising at least one selected from the group consisting of the Desulfovibrionaceae family strain, cultures thereof, culture mediums, and extracts obtained therefrom as an active ingredient, to an individual in need of administration.

Still another embodiment of the present invention relates to a food composition for preventing, alleviating or treating brain tumor in subjects with high glucose intake.

### CONCRETE DETAILS FOR IMPLEMENTATION OF THE INVENTION

Hereinafter, the present invention will be described in further detail by examples. It would be obvious to those skilled in the art that these examples are intended to be more concretely illustrative and that the scope of the present invention as set forth in the appended claims is not limited to or by the examples.

### EXAMPLES

### Preparation Example: Preparation of A Brain Tumor Mouse Model Administered with a High Glucose Beverage

In order to investigate the relationship between high glucose beverages and brain tumors, groups were divided as shown in Table 1 below, the diet was restricted, and this was maintained for 5 weeks. Normal water (NW) or high glucose water (HGW) containing 20% by weight of dextrose was fed before brain tumor induction. Thereafter, GL261 (2×10⁵ cell), a mouse-derived brain tumor cell line, was injected intracranially to induce brain tumors.

TABLE 1

| Group | Note |
|---|---|
| Control group (Control diet; CD) | Normal water diet (NW) |
| High glucose diet group (High | High glucose drinking water diet |
| glucose water; HGW or HG) | with 20% dextrose by weight |

### Example 1: Confirmation of Inhibitory Effect on Brain Tumor Growth by Ingesting High Glucose Beverages

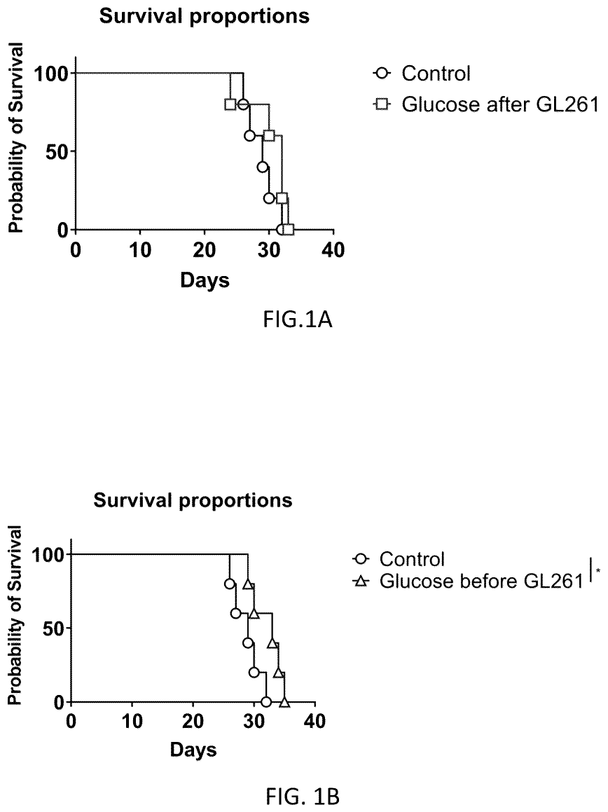

In order to investigate the effect of high glucose drinks on brain tumor survival rate, changes in survival rate and body weight were observed in the group of mice fed normal drinking water as a control group, mice fed a high-glucose drink after cancer transplantation, and mice fed a high-glucose drink for 5 weeks before cancer transplantation using brain tumor mouse model, and the results are shown in FIGS. 1A and 1B. As a result of the experiment, a significant increase in survival rate was observed in the group that continuously ingested high glucose beverages for 5 weeks before cancer transplantation (see FIGS. 1A and 1B).

### Example 2: Comparison of Brain Tumor Growth Inhibition According to High Glucose Beverage Intake Period

Since the experimental results in Example 2 above confirmed that continuous ingestion of high glucose beverages inhibits the growth of brain tumors, changes survival rates were compared with those of the control group after starting to ingest high glucose beverages 2 weeks or 4 weeks prior to transplantation to further determine whether taking the beverage for a certain period of time induces such changes.

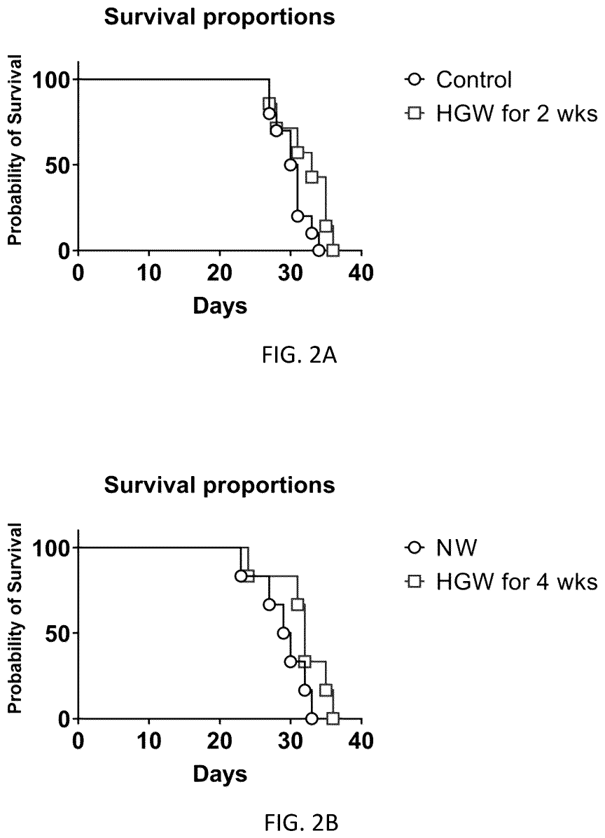

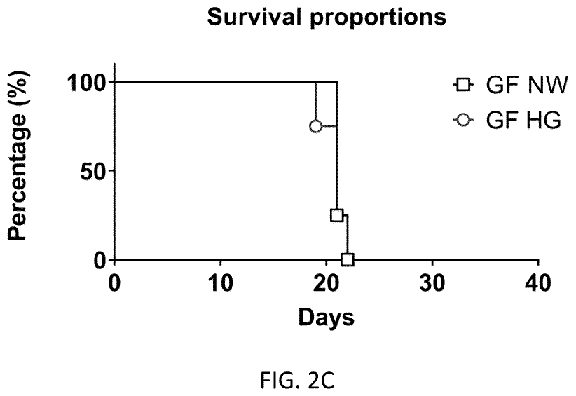

As a result of the experiment, a slight increase in survival rate was observed in the model ingesting 4 weeks prior compared to the model ingesting 2 weeks prior, and a significant increase was observed in FIG. 1B, which represents the results of ingesting 5 weeks prior, and thus it can be estimated that the longer the duration of continuous ingestion of the high glucose beverage, changes in the composition of symbiotic intestinal microorganisms have a positive effect on survival rate (see FIGS. 2A and 2B).

Furthermore, whether the brain tumor growth inhibition caused by continuous ingestion of high glucose beverages was due to intestinal microorganisms was further studied. To this end, an experiment was additionally conducted to inhibit brain tumor growth via high glucose beverages in germ-free mice without intestinal microorganisms grown by supplying sterile feed and drinking water in an external aseptic facility, and in this case, the consumption of high glucose beverages did not increase the survival rate due to brain tumors in germ-free mice, unlike before (see FIG. 2C). This suggests that the inhibition of brain tumor growth by consumption of high glucose beverages are caused by intestinal microorganisms.

Accordingly, the following experiment was conducted to confirm the composition of symbiotic intestinal microorganisms changed by the high glucose diet.

### Example 3: Analysis of Changes in Intestinal Microbial Community

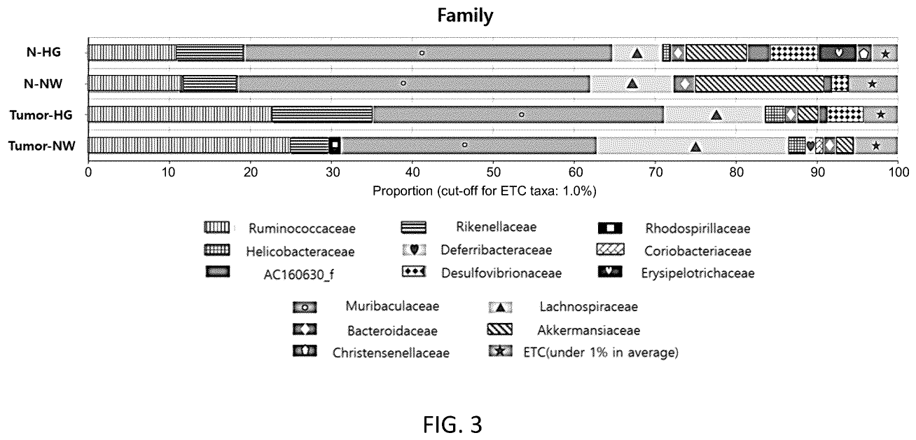

In order to confirm the composition of symbiotic intestinal microorganisms changed by the high glucose diet, DNA was extracted from the feces of the cancer-transplanted and non-transplanted groups in the high glucose beverage ingested model. 16S ribosomal DNA sequencing was performed on the extracted DNA to analyze the composition of the symbiotic intestinal microbial community. In order to find strains with significant differences in the above analysis, the characteristics of the microbial community that differentially increased or decreased at a specific time point were analyzed by using the linear discriminant analysis Effect Size (LEfSe) method. The results confirming the diversity of symbiotic intestinal microorganisms by group at the family level in the above manner are shown in FIG. 3.

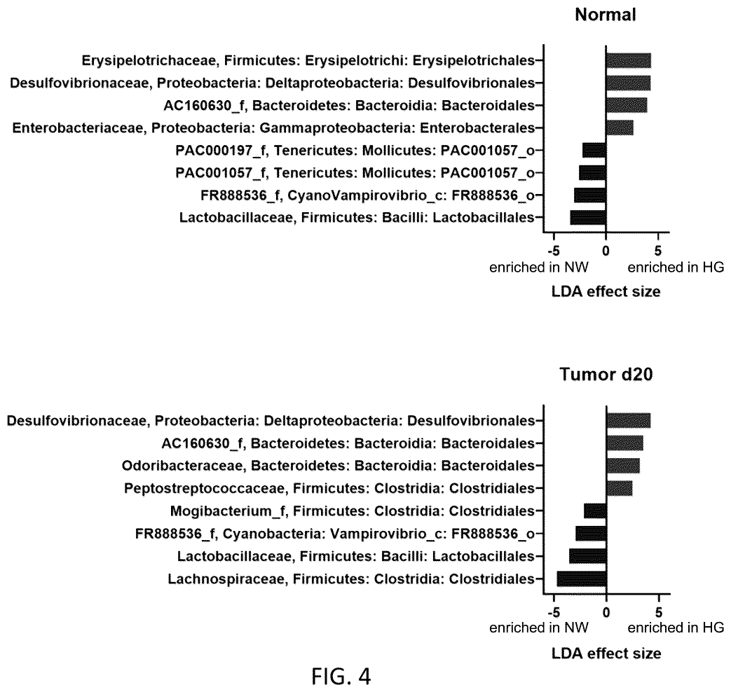

In addition, as a result of confirming the strains whose influence increased due to high glucose drinks in the LEfSe analysis in each of the cancer-transplanted and non-transplanted groups, strains of the Erysipelotrichaeae, Desulfovibrionaceae and AC160630_f family increased in the group without cancer transplantation, while strains of the Desulfovibrionaceae, AC160630_f and Odoribacteraceae family increased in the group with cancer transplantation (see FIG. 4). It was confirmed that the Desulfovibrionaceae strain increased due to the high glucose beverage regardless of the presence or absence of tumors. Thus, it can be seen that the survival rate increased due to the change in the composition of the strain, which was increased by ingesting the high glucose beverage.

Through this, it is suggested that the strain can be used as a strain for the prevention or treatment of brain tumors in a high glucose diet environment through a significant increase in the number of Desulfovibrionaceae family strains in the intestines of brain tumor mice fed a high glucose diet.

### Example 4: Verification of the Effect of Ingesting Strains of the Desulfovibrionaceae Family

Since it was confirmed that the consumption of high glucose beverages induces an increase in Desulfovibrionaceae family strains, in order to observe changes in survival rate caused by Desulfovibrio vulgaris bacteria belonging to the same family, the bacteria were obtained from the Korea Research Institute of Bioscience and Biotechnology's Korean Collection for Type Culture (KCTC), cultured. Then, a combination of 500 mg/L of ampicillin, 500 mg/L of gentamycin, 500 mg/L of metronidazole, 500 mg/L of neomycin sulfate, and 250 mg/L of vancomycin was administered to 4-week-old mice for 2 weeks for gut microbiota depletion. Thereafter, Desulfovibrio vulgaris bacteria was supplied to the mice through oral gavage 3 weeks a week, for a total of 4 weeks starting from 2 weeks prior to cancer transplantation until 2 weeks after. Separately, the experiment was conducted by supplying other mice with a high glucose beverage according to the 2-week bacteria administration period.

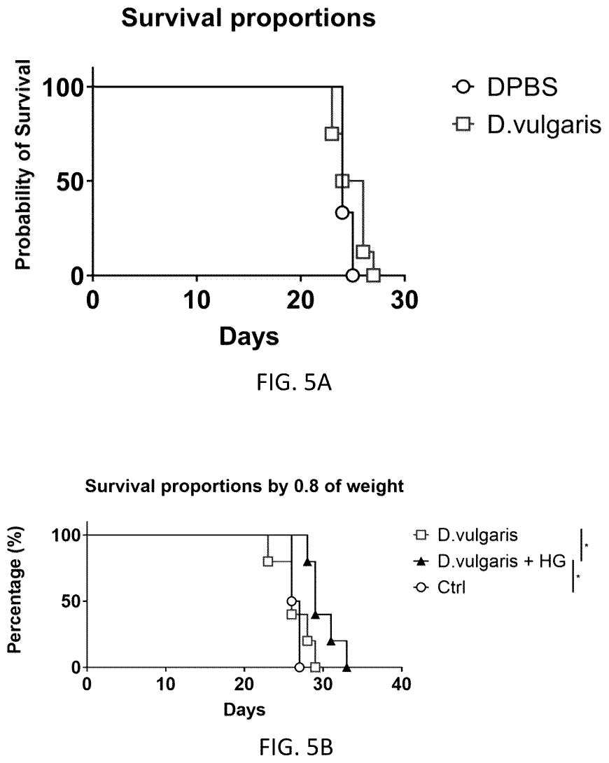

As a result of the experiment, while it was not possible to confirm a significant difference in the cancer survival rate by only ingesting bacteria, as shown in FIG. 5A, a significant increase in survival rate was confirmed in the mice supplied with a high glucose beverage according to the 2-week bacteria administration period (the group simultaneously supplied the high glucose beverage at the same time as the bacteria) (see FIG. 5B). Although consumption of high glucose beverages for a short period of about 2 weeks did not affect the survival rate, significant survival rates were observed when taking bacteria and high glucose beverages at the same time. This shows that high glucose is an essential condition for the survival rate increasing effect of Desulfovibrio vulgaris bacteria.

### Example 5: Single-Cell RNA Sequencing Analysis of Immune Cells in Brain Tumors

Since the Desulfovibrio vulgaris bacteria showed an anti-brain tumor effect in a high glucose diet environment, it was assumed that high glucose beverages would affect immune cells in the brain tumor microenvironment, and the effect on immune cells in the brain tumor microenvironment was studied to confirm the mechanism. Assuming that the change in survival rate due to the consumption of high glucose beverages was due to the influence of immune cells in brain tumors, single-cell RNA sequencing was performed to confirm the characteristics of immune cells in tumors. On the 20^{th} day after planting brain tumors in mice fed normal drinking water (NW) and high glucose drinking water (HG), immune cells were extracted from extracted brains, and single-cell RNA sequencing analysis was performed on the corresponding immune cells to analyze the characteristics of the immune cells. After extracting immune cells from brain tumors, Uniform Manifold Approximation and Projection (UMAP) analysis was performed after conducing single-cell RNA sequencing to confirm changes according to the consumption of high glucose beverages.

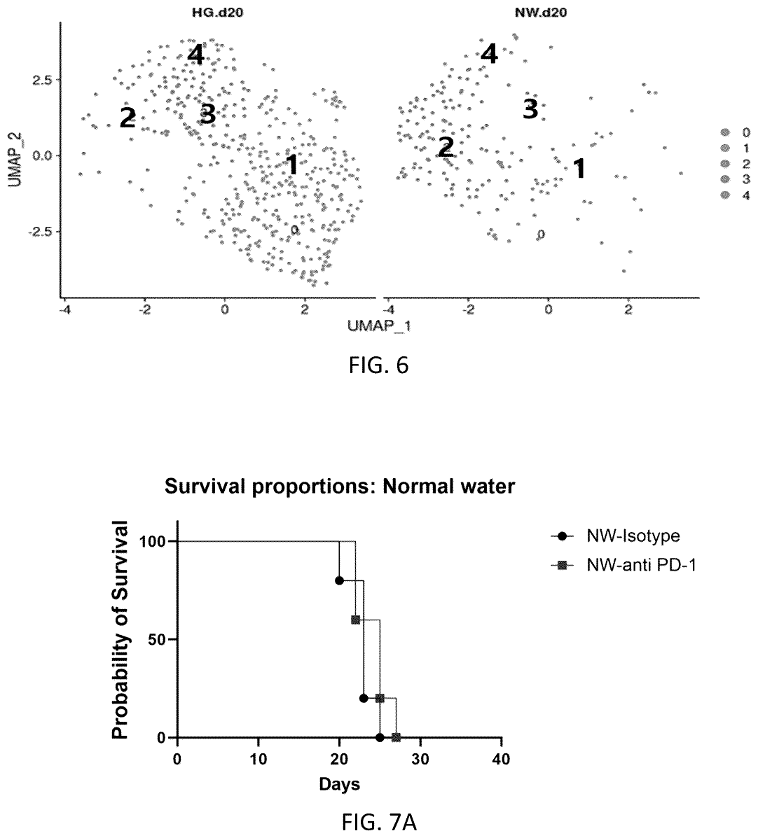

As a result of the experiment, it was confirmed that the population of CD8+ T cells increased when high glucose beverages were consumed, referring to FIG. 6, and that there was a difference in the form of the population between the group that consumed high glucose beverages (HG) and that consumed regular drinking water (NW). In other words, the immune cell transcriptome analysis shows that the number of CD8+ T cells in the brain tumors of mice fed a high glucose diet significantly increased, which means that T cell activation was induced.

### Example 6: Confirmation of Therapeutic Synergistic Effect with Immune Checkpoint Inhibitors

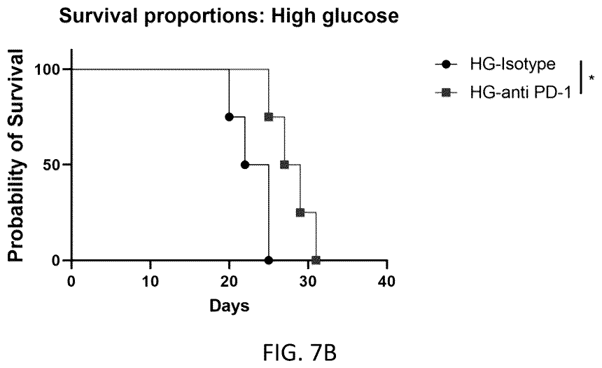

Since changes in CD8+ T cells caused by the consumption of high glucose beverages were confirmed, the effect of these changes on immunotherapy using anti-PD-1 antibodies targeting CD8+ T cells was studied. At 9, 12 and 15 days after cancer transplantation in the brain tumor mouse model, 200 ug of anti-PD-1 depletion antibody was diluted in 100 ul of Dulbecco's Phosphate-Buffered Saline (DPBS) and injected into the mice's peritoneal cavity, and the survival rates were compared and are shown in FIGS. 7A and 7B.

As a result of the experiment, while the survival rate of brain tumors did not improve despite intraperitoneal injection of anti-PD-1 in mice supplied with normal drinking water, as shown in FIG. 7A, the survival rate significantly increased as a result of intraperitoneal injection of anti-PD-1 in mice that consumed the high glucose beverage, as shown in FIG. 7B. Based on these results, it was confirmed that anti-PD-1 immunotherapy, which did not show a significant effect in the existing brain tumor model, enhanced the therapeutic effect and improved the survival of mice when combined with the consumption of high glucose beverages. This means that the Desulfovibrio vulgaris bacteria, one of the intestinal microorganisms in a high glucose diet environment, has a synergistic effect on the treatment of brain tumors with anti-PD-1 antibodies, an immune checkpoint inhibitor, and thus it is expected that it can greatly contribute to improving survival rate by improving brain tumor immunity.

Having described specific embodiments of the present invention in detail above, it is clear that these specific techniques are only preferred embodiments for those skilled in the art, and the scope of the present invention is not limited thereto. Accordingly, the scope of this invention is to be determined by appended claims and their equivalents.

### INDUSTRIAL APPLICABILITY

The composition of the present invention can prevent, alleviate or treat brain tumor very effectively by inducing changes in the composition of the gut microbiota, particularly changes in the composition of Desulfovibrionasaceae strains in a high glucose diet environment. Furthermore, it induces a synergistic effect with anticancer drugs, particularly immune checkpoint inhibitors, and thus it can ultimately contribute to improving the survival rate of brain tumor patients.

1-16. (canceled)

17. A method for preventing or treating a brain tumor comprising administering to a subject in need thereof, as an active ingredient, a pharmaceutically effective amount of a pharmaceutical composition comprising at least one selected from the group consisting of a Desulfovibrionaceae family strain, a culture thereof, a culture medium, and an extract obtained therefrom.

18. The method of claim 17, wherein the pharmaceutical composition is administered to a subject who has been on a high glucose diet.

19. The method of claim 18, wherein the subject on a high glucose diet is a subject who consumes more than 0.02 g/kcal as a ratio of a total daily sugar intake to a total daily caloric intake.

20. The method of claim 17, wherein the culture medium is one in which the Desulfovibrionaceae family strain is removed from the culture of the Desulfovibrionaceae family strain.

21. The method of claim 17, wherein the brain tumor is at least one selected from the group consisting of meningioma, pituitary tumor, hemangioblastoma, papilloma, glial cyst, glioma, oligodendrocyte glioma, glioblastoma, lymphoma, and metastatic brain tumor.

22. The method of claim 17, wherein the Desulfovibrionaceae family strain is at least one species selected from the group consisting of Desulfovibrio vulgaris (D. vulgaris), Desulfovibrio acrylicus (D. acrylicus), Desulfovibrio aerotolerans (D. aerotolerans), Desulfovibrio aespoensis (D. aespoeensis), Desulfovibrio africanus (D. africanus), Desulfovibrio alaskensis (D. alaskensis), Desulfovibrio alcoholivorans (D. alcoholivorans), Desulfovibrio alkalitolerans (D. alkalitolerans), Desulfovibrio aminophilus (D. aminophilus), Desulfovibrio arcticus (D. arcticus), Desulfovibrio barsi (D. baarsii), Desulfovibrio baculatus (D. baculatus), Desulfovibrio bastini (D. bastinii), Desulfovibrio biadensis (D. biadhensis), Desulfovibrio bizertensis (D. bizertensis), Desulfovibrio burkinensis (D. burkinensis), Desulfovibrio butyratiphilus (D. butyratiphilus), Desulfovibrio capillatus (D. capillatus), Desulfovibrio carbinolicus (D. carbinolicus), Desulfovibrio carbinoliphilus (D. carbinoliphilus), Desulfovibrio cuneatus (D. cuneatus), Desulfovibrio dechloroacetivorans (D. dechloracetivorans), Desulfovibrio desulfuricans (D. desulfuricans), Desulfovibrio perirreducens (D. ferrireducens), Desulfovibrio frigidus (D. frigidus), Desulfovibrio fructosivorans (D. fructosivorans), Desulfovibriofurfuralis (D. furfuralis), Desulfovibrio gabonensis (D. gabonensis), Desulfovibrio giganteus (D. giganteus), Desulfovibrio gigas (D. gigas), Desulfovibrio gracilis (D. gracilis), Desulfovibrio halophilus (D. halophilus), Desulfovibrio hydrothermalis (D. hydrothermalis), Desulfovibrio idahonensis (D. idahonensis), Desulfovibrio indonesiensis (D. indonesiensis), Desulfovibrio inopinatus (D. inopinatus), Desulfovibrio internalis (D. intestinalis), Desulfovibrio legallii (D. legallii), Desulfovibrio alitoralis (D. alitoralis), Desulfovibrio longreachensis (D. longreachensis), Desulfovibrio longus (D. longus), Desulfovibrio magneticus (D. magneticus), Desulfovibrio marinus (D. marinus), Desulfovibrio marinisediminis (D. marinisediminis), Desulfovibrio marrakechensis (D. marrakechensis), Desulfovibrio mexicanus (D. mexicanus), Desulfovibrio multispirans (D. multispirans), Desulfovibrio oceani (D. oceani), Desulfovibrio oxamicus (D. oxamicus), Desulfovibrio oxyclinae (D. oxyclinae), Desulfovibrio paquesii (D. paquesii), Desulfovibrio piezophilus (D. piezophilus), Desulfovibrio pigra (D. pigra), Desulfovibrio portus (D. portus), Desulfovibrio profundus (D. profundus), Desulfovibrio psychrotolerans (D. psychrotolerans), Desulfovibrio putealis (D. putealis), Desulfovibrio salixigens (D. salixigens), Desulfovibrio sapovorans (D. sapovorans), Desulfovibrio senezii (D. senezii), Desulfovibrio simplex (D. simplex), Desulfovibrio sulfodismutans (D. sulfodismutans), Desulfovibrio termitidis (D. termitidis), Desulfovibrio thermophilus (D. thermophilus), Desulfovibrio tunisiensis (D. tunisiensis), Desulfovibrio vietnamensis (D. vietnamensis), and Desulfovibrio zosterae (D. zosterae) belonging to a Desulphovibrio genus.

23. The method of claim 17, wherein the pharmaceutical composition further comprises an immune checkpoint inhibitor.

24. The method of claim 23, wherein the immune checkpoint inhibitor is at least one selected from the group consisting of a CTLA-4 receptor inhibitor, a PD-1 receptor inhibitor, a PD-L1 ligand inhibitor, a PD-L2 ligand inhibitor, a LAG-3 receptor inhibitor, a TIM-3 receptor inhibitor, a BTLA receptor inhibitor, and a KIR receptor.

25. The method of claim 23, wherein the immune checkpoint inhibitor specifically binds to at least one protein selected from the group consisting of CTLA-4, PD-1, PD-L1, PD-L2, LAG-3, TIM-3, BTLA, and KIR.

26. The method of claim 23, wherein the pharmaceutical composition induces an increase of the activity of T cells.

27. A method for preventing or alleviating a brain tumor comprising administering to a subject in need thereof, as an active ingredient, a food composition comprising at least one selected from the group consisting of a Desulfovibrionaceae family strain, a culture thereof, a culture medium, and an extract obtained therefrom.

28. The method of claim 27, wherein the food composition is administered to a subject who has been on a high glucose diet.

29. The method of claim 28, wherein the subject on a high glucose diet is a subject who consumes more than 0.02 g/kcal as a ratio of a total daily sugar intake to a total daily caloric intake.

30. The method of claim 27, wherein the culture medium is one in which the Desulfovibrionaceae family strain is removed from the culture of the Desulfovibrionaceae family strain.

31. The method of claim 27, wherein the brain tumor is at least one selected from the group consisting of meningioma, pituitary tumor, hemangioblastoma, papilloma, glial cyst, glioma, oligodendrocyte glioma, glioblastoma, lymphoma, and metastatic brain tumor.

32. The method of claim 27, wherein the Desulfovibrionaceae family strain is at least one species selected from the group consisting of Desulfovibrio vulgaris (D. vulgaris), Desulfovibrio acrylicus (D. acrylicus), Desulfovibrio aerotolerans (D. aerotolerans), Desulfovibrio aespoensis (D. aespoeensis), Desulfovibrio africanus (D. africanus), Desulfovibrio alaskensis (D. alaskensis), Desulfovibrio alcoholivorans (D. alcoholivorans), Desulfovibrio alkalitolerans (D. alkalitolerans), Desulfovibrio aminophilus (D. aminophilus), Desulfovibrio arcticus (D. arcticus), Desulfovibrio barsi (D. baarsii), Desulfovibrio baculatus (D. baculatus), Desulfovibrio bastini (D. bastinii), Desulfovibrio biadensis (D. biadhensis), Desulfovibrio bizertensis (D. bizertensis), Desulfovibrio burkinensis (D. burkinensis), Desulfovibrio butyratiphilus (D. butyratiphilus), Desulfovibrio capillatus (D. capillatus), Desulfovibrio carbinolicus (D. carbinolicus), Desulfovibrio carbinoliphilus (D. carbinoliphilus), Desulfovibrio cuneatus (D. cuneatus), Desulfovibrio dechloroacetivorans (D. dechloracetivorans), Desulfovibrio desulfuricans (D. desulfuricans), Desulfovibrio perirreducens (D. ferrireducens), Desulfovibrio frigidus (D. frigidus), Desulfovibrio fructosivorans (D. fructosivorans), Desulfovibriofurfuralis (D. furfuralis), Desulfovibrio gabonensis (D. gabonensis), Desulfovibrio giganteus (D. giganteus), Desulfovibrio gigas (D. gigas), Desulfovibrio gracilis (D. gracilis), Desulfovibrio halophilus (D. halophilus), Desulfovibrio hydrothermalis (D. hydrothermalis), Desulfovibrio idahonensis (D. idahonensis), Desulfovibrio indonesiensis (D. indonesiensis), Desulfovibrio inopinatus (D. inopinatus), Desulfovibrio internalis (D. intestinalis), Desulfovibrio legallii (D. legallii), Desulfovibrio alitoralis (D. alitoralis), Desulfovibrio longreachensis (D. longreachensis), Desulfovibrio longus (D. longus), Desulfovibrio magneticus (D. magneticus), Desulfovibrio marinus (D. marinus), Desulfovibrio marinisediminis (D. marinisediminis), Desulfovibrio marrakechensis (D. marrakechensis), Desulfovibrio mexicanus (D. mexicanus), Desulfovibrio multispirans (D. multispirans), Desulfovibrio oceani (D. oceani), Desulfovibrio oxamicus (D. oxamicus), Desulfovibrio oxyclinae (D. oxyclinae), Desulfovibrio paquesii (D. paquesii), Desulfovibrio piezophilus (D. piezophilus), Desulfovibrio pigra (D. pigra), Desulfovibrio portus (D. portus), Desulfovibrio profundus (D. profundus), Desulfovibrio psychrotolerans (D. psychrotolerans), Desulfovibrio putealis (D. putealis), Desulfovibrio salixigens (D. salixigens), Desulfovibrio sapovorans (D. sapovorans), Desulfovibrio senezii (D. senezii), Desulfovibrio simplex (D. simplex), Desulfovibrio sulfodismutans (D. sulfodismutans), Desulfovibrio termitidis (D. termitidis), Desulfovibrio thermophilus (D. thermophilus), Desulfovibrio tunisiensis (D. tunisiensis), Desulfovibrio vietnamensis (D. vietnamensis), and Desulfovibrio zosterae (D. zosterae) belonging to a Desulphovibrio genus.
